(19) United States
(12) Patent Application Publication
(10) Pub. No.: US 2015/0216468 A1
(43) Pub. Date: Aug. 6, 2015

(54) METHOD AND SYSTEM FOR REAL-TIME INSIGHT DETECTION USING EEG SIGNALS

(71) Applicant: KONICA MINOLTA LABORATORY U.S.A., INC., San Mateo, CA (US)

(72) Inventors: Michel Vidal-Naquet, San Mateo, CA (US); Hideki Tanaka, Fuchu (JP); Haisong Gu, Cupertino, CA (US)

(73) Assignee: KONICA MINOLTA LABORATORY U.S.A., INC., San Mateo, CA (US)

(21) Appl. No.: 14/169,728

(22) Filed: Jan. 31, 2014

(51) Int. Cl. A61B 5/00; A61B 5/0476

(52) U.S. Cl. CPC A61B 5/4064; A61B 5/0476; A61B 5/4076

(57) ABSTRACT

A method, system and computer readable medium for detecting creativity in real-time are disclosed. The method includes sensing electrical activity along a scalp of a subject during a learning phase, the learning phase including presenting to the subject one or more tasks configured to generate electrical activity corresponding to cortical events that are likely to correspond to a creative type experience and cortical events that are likely to correspond to a non-creative type experience. Features from the electrical activity obtained during the learning phase are extracted to create a brainwave profile for the subject. Real-time electrical activity along the scalp of the subject is sensed during a performance of one or more real-time tasks, and the electrical activity of the subject is compared to previously recorded electrical activity using the brainwave profile for the subject to classify the electrical activity obtained during the performance of the one or more real-time.

### FIELD OF THE INVENTION

The present disclosure relates to a method and system for real-time insight detection using EEG signals, and wherein the method and systems can be applied to monitor real-time human insight events.

### BACKGROUND

There is a growing interest in using neural signals measured from the Human working brain to interact with machines and understand the brain state of people. For example, knowing the brain state of a person during working hours can provide information on work performance at any given time, and enable the leveraging of specific periods at which the worker is likely to be particularly productive or insightful.

In addition, knowledge workers can be required to solve creative problems and similar tasks. To understand creative processes leading to insight, “Eureka”, or “Aha” moments, some psychology research groups perform ensemble analysis of neural signals collected after an experiment and attempt to characterize the neural signatures of creativity. Such approaches to characterize creativity, “Aha” and similar moments are not performed in real time. However, it will be useful to have a system that can detect creativity events in real time, since such a system can help determine directions for improved creativity and leveraging creative moments when they occur.

### SUMMARY

In consideration of the above issues, it would be desirable to have a method and system, which can enable the detection of insight events in real-time occurring for a worker wearing a consumer level EEG recording device is disclosed. For example, research has demonstrated that some EEG frequencies of particular channels can differentiate between “insight” or “creativity”, and “analysis” or “non-creative′” brain states leading to problem solutions. In accordance with an exemplary embodiment, the method and system can use these scientific results for detecting, in real time, cortical events that are likely to correspond to insight type solutions to problems, for example, solutions with high value.

In accordance with an exemplary embodiment, a system and method for the detection of creativity events occurring in the brain, from brain signals, and in real time is disclosed. For example, in accordance with an exemplary embodiment, the system disclosed uses a machine learning approach that learns time-frequency signatures of creativity based on features such as Fourier components or wavelets. In accordance with an exemplary embodiment, the system can be trained to classify, in real time, the feature vector extracted from recorded neural signals into “insight” or “non-insight” types of events. The system can also be trained to detect, for example, when a person is in a mental state likely to lead to insight.

In accordance with an exemplary embodiment, a method for detecting creativity in real-time is disclosed, the method comprising: sensing electrical activity along a scalp of a subject during a learning phase, the learning phase including presenting to the subject one or more tasks configured to generate electrical activity corresponding to cortical events that are likely to correspond to a creative type experience and cortical events that are likely to correspond to a non-creative type experience; classifying the electrical activity obtained during the learning phase to create a brainwave profile for the subject; sensing in real-time electrical activity along the scalp of the subject during a performance of one or more real-time tasks; comparing the electrical activity of the subject to previously recorded electrical activity using the brainwave profile for the subject; and classifying the electrical activity obtained during the performance of the one or more real-time tasks into the cortical events that are likely to correspond to the creative type experience and the cortical events that are likely to correspond to the non-creative type experience.

In accordance with an exemplary embodiment, a system for detecting creativity in real-time is disclosed, the system comprising: a sensing device for sensing electrical activity over one or more frequency bands along a scalp of a subject, the sensing device configured to: sense electrical activity along a scalp of a subject during a learning phase, the learning phase including presenting to the subject one or more tasks configured to generate electrical activity corresponding to cortical events that are likely to correspond to a creative type experience and cortical events that are likely to correspond to a non-creative type experience; and sense in real-time electrical activity along the scalp of the subject during a performance of one or more real-time tasks; and a computer device having executable instructions for: classifying the electrical activity obtained during the learning phase to create a brainwave profile for the subject; comparing the electrical activity of the subject to previously recorded electrical activity using the brainwave profile for the subject; and classifying the electrical activity obtained during the performance of the one or more real-time tasks into the cortical events that are likely to correspond to the creative type experience and the cortical events that are likely to correspond to the non-creative type experience.

In accordance with an exemplary embodiment, a non-transitory computer readable medium containing a computer program having computer readable code embodied for detecting creativity in real-time is disclosed, comprising: sensing electrical activity along a scalp of a subject during a learning phase, the learning phase including presenting to the subject one or more tasks configured to generate electrical activity corresponding to cortical events that are likely to correspond to a creative type experience and cortical events that are likely to correspond to a non-creative type experience; classifying the electrical activity obtained during the learning phase to create a brainwave profile for the subject; sensing in real-time electrical activity along the scalp of the subject during a performance of one or more real-time tasks; comparing the electrical activity of the subject to previously recorded electrical activity using the brainwave profile for the subject; and classifying the electrical activity obtained during the performance of the one or more real-time tasks into the cortical events that are likely to correspond to the creative type experience and the cortical events that are likely to correspond to the non-creative type experience.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a schematic illustration of a user with an EEG recording headset and a computer in accordance with an exemplary embodiment;

FIG. 2 is a flow chart showing a learning part of a method and system for insight detection in accordance with an exemplary embodiment;

FIG. 3 is a flow chart showing a classification of signals of a method and system for insight detection in accordance with an exemplary embodiment;

FIG. 4 shows a ROC (Receiver Operation Characteristics) curve for a specific channel (for example, P7) and a specific user;

FIG. 5 shows an application of threshold classifiers for time intervals during a work period;

FIG. 6 shows a full workflow, wherein the full workflow combines the learning phase described in FIG. 2, and the detection phase, described in FIG. 3, and wherein the detection phase is repeated until it is deemed necessary to go through a new learning phase; and

FIG. 7 shows a flow chart of a machine learning system in accordance with an exemplary embodiment.

### DETAILED DESCRIPTION

Reference will now be made in detail to the embodiments of the invention, examples of which are illustrated in the accompanying drawings. Wherever possible, the same reference numbers are used in the drawings and the description to refer to the same or like parts.

This disclosure relates to an electroencephalograph (EEG) based system and method for insight detection, which includes a method and apparatus for determining appropriate EEG channels to record from and extracting basic statistics for learning classification models. In accordance with an exemplary embodiment, the method and system as disclosed can be applied during a “learning stage”, and application of the insight event detector during a work or work environment. Once a determination of the insightful or creative mental status can be achieved as disclosed herein, the mental status of the subject during certain time or event can be leveraged to increase production or performance, for example, in a work environment. For example, in accordance with an exemplary embodiment, a cost analysis of activities or production obtained from of the subject during a creative state can be performed versus activities or production obtained from the subject during non-creative mental states of the subject.

As used herein biological activity relates to the study of life and living organisms, which can include biological activities such as perspiration or sweat, heart rate, nervous signals, brain waves, and/or combinations thereof. The biological activities can be measured with any suitable sensing devices, which can detect changes in biological activity of a subject. For example, brainwaves can be measured and/or detected using an EEG, wherein the EEG includes one or more electrodes, which detect voltage fluctuations resulting from the combined electrical signaling activity of neurons within the brain.

In accordance with an exemplary embodiment as disclosed herein, for example, “insight” can occur when a person suddenly reinterprets a stimulus, situation, or event to produce a nonobvious, non-dominant interpretation. For example, this can take the form of a solution to a problem (an “aha moment”), comprehension of a joke or metaphor, or recognition of an ambiguous percept.

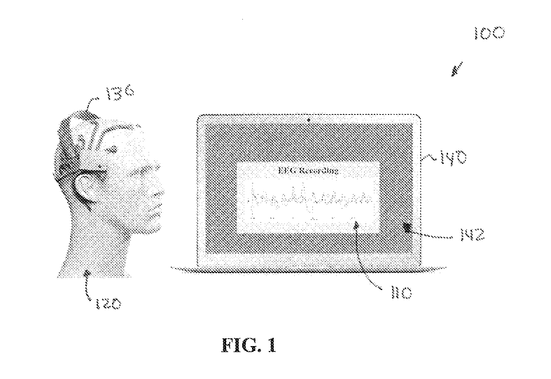

FIG. 1 illustrates a system 100, which uses EEG data 110 collected from a subject and/or user 120 for detection of insight of an individual's mental state, and wherein based on the mental insight, analysis of creativity can be performed in real-time in accordance with an exemplary embodiment. As shown in FIG. 1, the system 100 can include a brain wave recording device (or headset) 130, which can be an EEG recording device or headset, which generates EEG data 110. In accordance with an exemplary embodiment, the headset 130 preferably is a user-friendly device, which is commercially available.

In accordance with an exemplary embodiment, an estimation of the mental state of the subject 120 is generated from the EEG data 110. In accordance with an exemplary embodiment, the EEG recording device 130 can be any suitable EEG recording devices, from a relatively inexpensive commercial device with only one electrode to more advanced medical-grade devices with dozens of electrodes. For example, in accordance with an exemplary embodiment, the brainwave sensing device 130 can be an EEG recording device having a plurality of sensors, which can obtain a plurality of signals, which can be analyzed to information about an individual's mental state. In accordance with an exemplary embodiment, the brain wave sensing device 130 can be a single-electrode EEG device, which can achieve relatively good accuracy at classifying mental states as disclosed herein. For example, in accordance with an exemplary embodiment, commercial EEG detection devices, which can be easier to use, can be effective for the method and system as disclosed herein.

In accordance with an exemplary embodiment, the brainwave sensing device 130 is a device, which records electrical activity along the scalp. For example, the brainwave sensing device 130 can be an electroencephalography (EEG), which measures voltage fluctuations resulting from the combined electrical signaling activity of neurons within the brain. An EEG employs one or more electrodes on the scalp to measure this electrical activity, which is often rhythmic or oscillatory in nature. The rhythmic activity can be analyzed in terms of activity in different frequency bands, which are extracting using spectral methods. However, in accordance with an exemplary embodiment, any type of device, which senses electrical activity along the scalp, can be used.

For example, an EEG device can use a plurality of electrodes, which detect the voltage fluctuations. The plurality of electrodes can be in the form of a cap or netting, which places the plurality of electrodes in one or more desired location along the scalp. Alternatively, the electrode can be positioned on the subject individually or by any suitable manner. In addition, touchless sensors or other sensors, which do not make contact with the scalp of the subject, can be used to detect the electrical activity.

The electrical activity from the brainwave sensing device 130 can be transmitted via a connection or wire to a computer or processing system 140. The sensing device 130 can alternatively include wireless technology, which transmits the data or electrical activity obtained from the subject to the data processing system 140 without a hard connection or wire. The brainwave sensing device 130 senses brainwave signals of a subject, which can be recorded over one or more frequency bands. It can be appreciated that there are a variety of affordable devices with only one electrode to dozens of electrodes. Although more sensors can detect more information, even one electrode can be used to detect insight.

In accordance with an exemplary embodiment, the computer or processing device 140 preferably has a memory, a processor, an operating system and/or software and/or an optional graphical user interface (GUI) and/or display. It can be appreciated that the computer or processing device 140 can be a standalone computer, or can be contained within one or more computer devices, wherein each of the one or more computer devices has a memory, a processor, an operating system and/or software, and a graphical user interface (GUI) or display.

In accordance with an exemplary embodiment, the computer or processing device 140 can include a computer monitor & keyboard, for stimuli presentation and user input during learning phase, and a computer used for stimuli generation, recording of the data, learning of parameters for classification, and actual classification. In accordance with an exemplary embodiment, the computer or processing device 140 preferably includes software components, which can perform stimuli generation for learning phase and computation of signal statistics during learning phase, and/or classification of signals during classification/working phase.

For example, for an EEG, the frequency bands recorded can be between approximately 0.1 hertz (“Hz”) to approximately 100 hertz (Hz). In accordance with an exemplary embodiment, each of the frequency bands can be generally classified within one or more frequency bands. For example, frequencies of 4 hertz (Hz) or lower can be assigned to the delta (Δ) band, from approximately 5 Hz to 7 Hz to the theta (8) band, from approximately 8 Hz to 15 Hz to the alpha (α) band, from approximately 16 Hz to 24 Hz to the beta (β) band, and from approximately 24 Hz to 40 Hz to the gamma (γ) band. However, the frequencies are only exemplary and different ranges can be used to define a frequency band in accordance with functional and behavioral properties. For example, delta waves are usually associated with the deepest stages of sleep (3 and 4 NREM), also known as slow-wave sleep (SWS), and aid in characterizing the depth of sleep.

In accordance with an embodiment, the EEG-based insight detection method can include the placement of one or more electrodes on the scalp of the subject using an electrode cap or netting. Alternatively, the electrodes can be individually placed on the scalp of the subject as desired.

In accordance with an example, the signal extraction for the EEG can be the raw voltage fluctuation (μV) signal between approximately 0 and 100 Hz within a defined or set period of time (for example, approximately 10 minutes to 2 hours during the work day), which is collected by the EEG device or headset 130. The raw signals can be transferred into the frequency domain by FFT (Fast Fourier Transform) and divided into one or more frequency bands. For example, the one or more frequency bands can be defined as delta (A), four or less Hz, theta (θ) approximately 5 to 7 Hz, alpha (α) approximately 8 to 15 Hz, beta (β) approximately 16 to 24 Hz, and gamma (γ) approximately 24-40 Hz. In accordance with another aspect, the one or more frequency bands can be defined delta (Δ), less than 4 hertz (“Hz′)), theta (θ) approximately 4 to 8 Hz, alpha (α) approximately 8 to 13 Hz, beta (β) approximately 13-30 Hz, and gamma (γ) approximately 30 to 100 Hz. The spectrum can also be obtained as the power of each of the one or more frequency bands.

In accordance with an exemplary embodiment as shown in FIG. 1, the EEG data 110 is recorded from a subject using a brain wave sensing device 130, and an estimate of mental state of the subject 120 can be made. For example, in accordance with an exemplary embodiment, the system includes: (1) a set of mental state dimensions (for example, creativity, focus, relaxation) by which to characterize an individual's mental state; (2) an insight detection list, with each insight detection list indicating what levels of each mental state dimension; and (3) an EEG recording device.

In accordance with an exemplary embodiment, the method and system for EEG-based insight detection can consist of a mental state estimation phase, which can be based on a biological signal, for example, an EEG signal, which can be used by a machine learning-based algorithm to estimate mental states of the subject, an insight detection process, wherein the estimated predicted mental state is matched against the requirements of different insight event detections.

In accordance with an exemplary embodiment, a mental state estimation can either be done using either a rule-based method, where a method and/or process based a determination by an estimation of mental states to pre-defined features of the EEG signal map onto different mental state dimensions or insight, or alternatively, a learning based method, where the mapping is learned from previously collected EEG data that is collected and/or used during actual activities or diagnostic testing by one or more subjects. In accordance with an exemplary embodiment, by optimizing insight detection according to an individual's current mental state, the individual's overall productivity and insight detection can improve.

In accordance with an exemplary embodiment, for example, EEG signals can be recorded when a subject is performing a set of diagnostic tasks or problem solving, for example, a compound remote association, such as described by Bowden & Jung-Beeman. The EEG data can then be analyzed by dividing the EEG signal into a plurality of windows or segments. For example, each of the plurality of windows or segments can be 0.5 seconds, 1.0 seconds, 2.0 seconds, 3.0 seconds, 4.0 seconds, 5.0 seconds, greater than 5.0 seconds and less than 10 seconds for further analysis.

In accordance with an exemplary embodiment, various features can be computed from the windowed EEG signal to help estimate and/or determine the subject's mental state for insight detection. For example, one of the various features used in EEG analysis are the power in pre-defined frequency bands, which include:

Delta—deep, non-REM sleep

Theta—Intuitive, creative, imaginative, dreaming

Alpha—Relaxed (but not drowsy)

Beta—Focused, thinking, alert

Gamma—Motor functions, higher mental activity.

In accordance with an exemplary embodiment, the signals or power in the frequency bands can be obtained by performing a fast Fourier transform (FFT) on the windowed EEG signal from each channel. In addition, besides the frequency band powers, other features can be used to distinguish mental states, for example, can include signal entropy, spectral entropy, and/or standard deviation. For example, if a device with multiple electrodes is being used, additional features such as the coherence or mutual information between pairs of electrodes can also be used. In accordance with an exemplary embodiment, each of the selected features can be concatenated into a vector, and this feature vector can be used to represent that window of EEG signal during mental state classification.

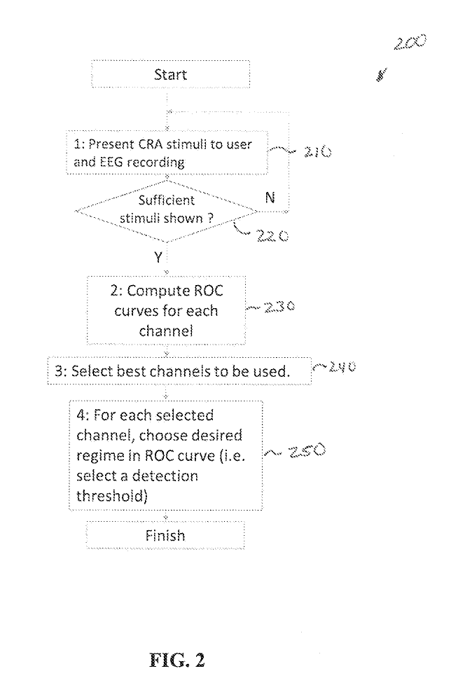

FIG. 2 is a flow chart 200 showing the learning part of a system and method for insight detection in accordance with an exemplary embodiment. As shown in FIG. 2, in step 210, compound remote association (CRA) tasks can be presented to the user. In accordance with an exemplary embodiment, CRA stimuli can be generated, for example, by a computer 140 and presented on a monitor 142 associated with the computer 140 to the user 120. The user 120 tries to solve the problem, orally saying the answer to a helper, or directly on the computer, and mentioning whether the solution was reached by “analysis” or “insight”. As shown in FIG. 2, in step 220, a determination is made if sufficient stimuli are shown based on the CRA tasks. If not, the process returns to step 210, wherein user continues to receive CRA tasks. However, if sufficient stimuli are shown, the process continues to step 230.

In step 230, after a sufficient number of trials has been reached, Receiver Operation Characteristic (ROC) Curves can be computed for a threshold classifier applied to the power of all channels of interest, within a certain time frame of the trial and for a frequency band of interest (for example, approximately 9 to 10 Hz or less than 30 Hz).

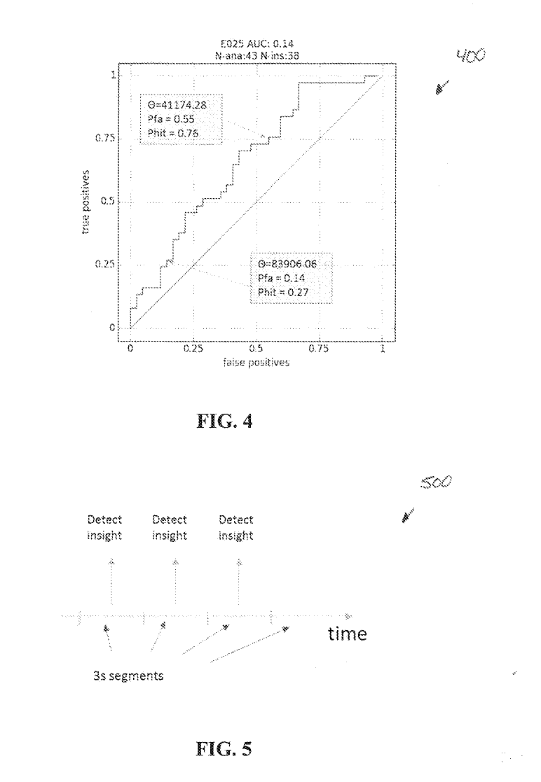

In accordance with an exemplary embodiment, an example of an ROC curve 400 for a specific channel and specific frequency band computed after a set of trials is shown in FIG. 4 (blue curve). For each channel, and each set of frequency bands of interest, the Area under the Curve (AUC) can be computed, which represents the performance of the classifier. In accordance with an exemplary embodiment, in step 240, the channel and frequency band that provides highest AUC can then be chosen for the user.

For each of the selected channels and bandwidths in step 250, a particular detection threshold can be selected based on the desired probabilities of True-Positives and False-Positives. This threshold can be used for that particular channel and bandwidth during the detection stage. In accordance with an exemplary embodiment, the formula for a threshold classifier can be given in the following equation:

$$\{ \begin{matrix}INS & if α \geq θ \\ ANA & if α < θ\end{matrix}$$

where α is the power of the channel within a frequency band of interest, and θ is the selected threshold. INS mean ‘insight’ is detected about the trial, and ANA means ‘analysis’ is detected. In accordance with an exemplary embodiment, the equation can provide a classification formula for the threshold classifier. If the power (α) is above a threshold θ, the signal is classified as “insight” (INS), otherwise it is classified as “analysis” (ANA).

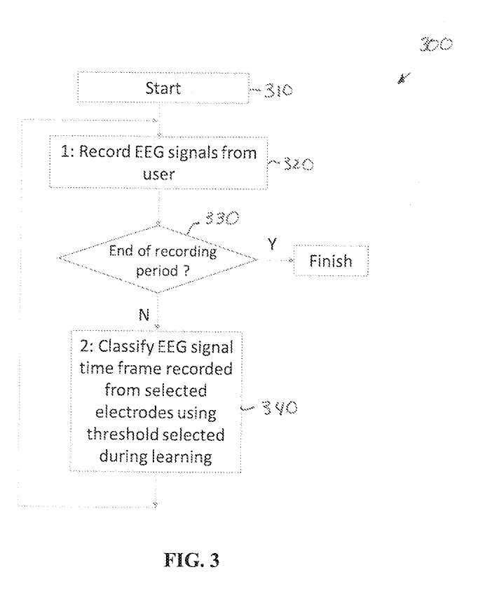

FIG. 3 is a flow chart 300 showing the classification of the signals for a method and system of insight detection in accordance with an exemplary embodiment. As shown in FIG. 3, the process starts in step 310, wherein for example, during a specified time frame or period, for example, work time, in step 320, EEG signals are recorded from the user 120 using a brain wave device 130, for example, a headset and computer 140, and the raw signals for each channel are recorded and filtered by the computer.

In step 320, the insight detection method determines, if the end of the recording period has been reached. If the end of the recording period has not been reached, the EEG signals continue to be recorded. If the end of the recording period is reached, in step 330, the recording process ends, and the data is forwarded to a computer or processing device 140 for analysis. In step 340, for each consecutive time segment of the signals, a software algorithm hosted on a computer or processing device 140, can apply previously selected detection thresholds on the recorded channels and bandwidths of interest in order to detect ‘insight’ events 500, as illustrated in FIG. 5. For example, the times at which ‘insight’ has been detected can be correlated with the particular activities the user was performing, helping attributing more value to those particular times.

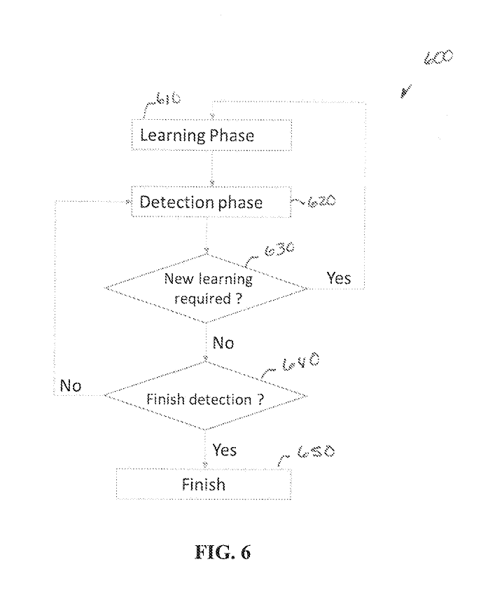

FIG. 6 shows a workflow 600, wherein the workflow combines the learning phase (step 610) described in FIG. 2, and the detection phase (step 620) described in FIG. 3, and wherein the detection phase is repeated until it is deemed necessary to go through a new learning phase 630. As shown in FIG. 6, the workflow can be configured to combines both learning and detection stages. In accordance with an exemplary embodiment, the learning phase can be performed anytime it is deemed required, for example, when the headset has moved, or if there was a break between working sessions and so on. In addition, for example, it may be determined in step 630, that the learning phase 610 should be repeated, the method and system could repeat the learning 610 and the detection phases 620 as disclosed for the subject 120. Once a final detection is determined in step 640, the process ends (step 650).

In accordance with an exemplary embodiment, the classifier as determined during the classification process 340, can be more sophisticated than a simple threshold function. For example, in accordance with an exemplary embodiment, a support vector machine can be applied to different frequency bands and different channels, a neural network, and/or a boosting based classifier. In addition, the learning does not have to be restricted to the threshold, and can depend on a specific classifier used.

In accordance with an exemplary embodiment, the learning stage can provide the parameters necessary for the classifier to detect insight events with best possible performance. For example, in accordance with an exemplary embodiment, the method to select appropriate channels and bandwidths can rely on any useful classification performance metric that can be derived from classifier false positive rate, true positive rate and number of class samples. For example, rather than using the AUC, the method can be based on mutual information, or a combination of precision and recall values.

In accordance with an exemplary embodiment, an algorithm is disclosed for deciding whether a new learning phase 610 is required or not, which can consist of a learning part and a decision part. In accordance with an exemplary embodiment, during the learning phase 610, the average power of a frequency band of interest can be computed, for example the alpha band. Denote by P this power. The computation of P can be based on the Fourier Transform of the recorded EEG signal during the whole learning period, or during segments of the period and averaged, or using similar methods. Importantly, the time period on which the power P is computed should be significantly larger than the time window used for classification of EEG signals into insight or analysis. For example, P could be computed over the whole training period or from time segments of 1 mn, 10 mn or 20 mn. This is in contrast to the 3 s period used to classify the EEG signal.

In accordance an exemplary embodiment, the decision part can include computing during the detection phase 620, a power Q of the same band of interest, using a temporal window that is also significantly longer the classification period, for example, 2 mn, or 5 mn. In step 630, Q to P is compared, and if the absolute difference between the two signals is larger than a pre-specified percent or ratio, (for example, 10%), noted r, then it is deemed that a new learning is required and/or should be performed.

In accordance with an exemplary embodiment, a third part can be performed prior to the learning and decision parts (parts 1 and 2), which can include the determination of a useful value of r. For example, this value could be determined also during the learning phase 610, or by any other appropriate method.

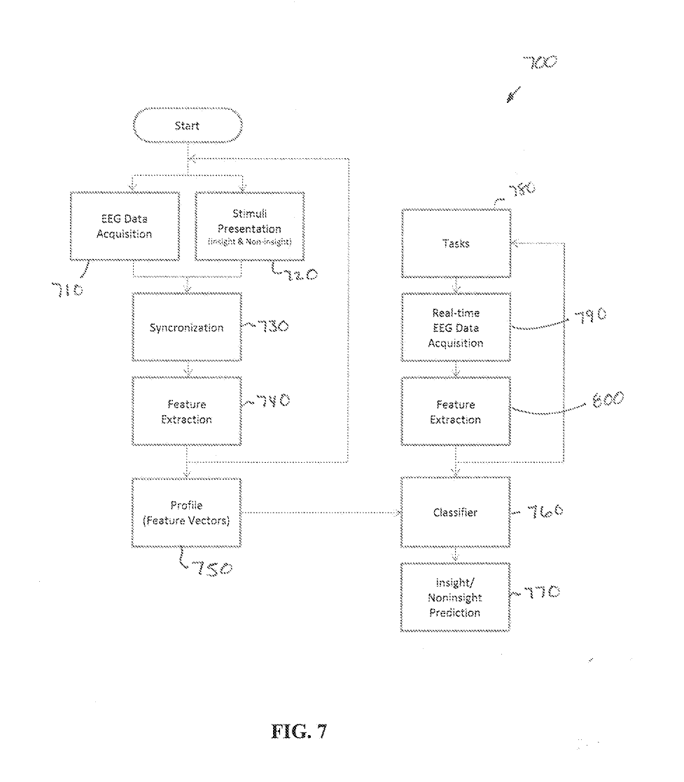

FIG. 7 shows a flow chart 700 of a machine learning system in accordance with an exemplary embodiment. As shown in FIG. 7, the process starts with an acquisition of EEG data 710 based on a presentation of stimuli 720. The presentation of stimuli 720 is designed to determine or classify the insight and non-sight thought or mental processes of the subject 120. In step 730, the acquired EEG data 710 and the presentation of stimuli 720 can be synchronized and in step 740, a feature vector can be extracted from the EEG data 710 and stimuli 720. In step 750, a profile for the subject 120 can be generated using one or more feature vectors. In step 760, the one or more feature vectors can be classified, for example, into “insight” or “creativity” mental processes, and “non-insight” or “non-creative” mental processes.

In accordance with an exemplary embodiment, the method and system as disclosed herein can be used in real-time for analysis of a subject thought process to help identify moments, which can have more insight than other moments. For example, in accordance with an exemplary embodiment, the subject 120 can perform a task 780, during which EEG data can be obtained in real-time (step 790) and an analysis thereof can be performed to generate an extraction of a feature vector (step 800) in real-time. The extracted feature vector can then be classified based on the classifier 760, which can generate a real-time prediction of the insight or non-insight 770 of the subject 120 during the performance of the task 780.

In accordance with an exemplary embodiment, several methods can be used for feature extraction in order to create feature vectors of good quality in the “learning phase”, and for feature extraction in the “detecting phase” in order to classify if a detection of insightful moment, for example, a “Aha” moment, occurs for a single task (real-time detection), either before the problem is solved, and/or when the problem is solved.

In accordance with an exemplary embodiment, to build a real-time detection system with machine-learning methods, it is necessary to generate for each user or subject 120, a brainwave profile (with feature vectors). In accordance with an exemplary embodiment, EEG signals can be recorded when a user 120 is performing diagnostics with insight and non-insight process. Stimuli presentations can be used as the diagnostics. In accordance with an exemplary embodiment, a sufficient amount of feature vectors is preferably and/or may be necessary for optimal performance of the machine algorithm.

In accordance with an exemplary embodiment, the presentation of stimuli to the subject during the learning phase can be performed using a stimuli that can be generated signals that can be detected by the brainwave sensing device or headset 130. For example, one type of stimuli called compound remote associates (Bowden & Jung-Beeman, 2003) can be used as a stimuli presentation. For example, the compound remote associate (CRA) stimulus asks “what word can form a familiar compound word or phrase with the each of these words?” pine, crab, sauce->apple (answer). The subject is then asked whether the solution or answer was based on “analysis” or “insight”.

In accordance with an exemplary embodiment, the method and system as disclosed herein can be configured to run on two programs (e.g., synchronization). The first program (program “A”) generates the stimuli and captures events such as user key presses and new stimulation presentation. The second program (program “B”) can run on the same or on a different computer, and records the brain signal data as well as the user events captured by the first program. In accordance with an exemplary embodiment, the two programs communicate using the serial port (e.g. COM port on Windows) as follows: Program A writes to the Serial port whenever an event such as user key press or a new stimulus is shown occurs. Program B regularly reads on the Serial port at a frequency that is similar the sampling frequency that the brain signal data is being recorded. If any data has been written by program “A”, program “B” will immediately record the event, such that the event is recorded synchronously with the brain signal data.

Once the brainwave profile is created and a learning stage is completed, the creativity can be detected in real time (detection stage, algorithms). In accordance with an exemplary embodiment, EEG data can be acquired and extracted to obtain a feature vector when the user performs a task. In accordance with an exemplary embodiment, the classifier can determines creativity state. For example, the machine learning algorithm can be dependent on the nature of the mental state dimensions. If the mental state dimensions are binary (e.g. creativity is either “yes” or “no”), a support vector machine (SVM) classifier can be one of the best methods. For example, for a standard linear SVM, a vector w and a constant b from the brain profile is determined in order to create the decision rule of the following form:

$$CREATIVITY = \{ \begin{matrix}YES , & w * {x}_{i} - b < 0 \\ NO , & w * {x}_{i} - b \geq 0\end{matrix}$$

where xᵢ is a feature vector. The SVM is a maximum-margin classifier; that is, the terms w and b can be learned in such a way as to maximize the distance from the hyperplane w*x−b=0 to the closest points on either side of it. On the other hand, if the mental state dimensions have multiple options (e.g., attention is rated on a scale of 1-5), a regression model such as Gaussian Process Regression can be used to output a predicted value.

In accordance with an exemplary embodiment, the classification stage can be performed by any classification function deemed useful for the task at hand, not only limited to the SVM algorithm. For example, several methods can be used for feature extraction in order to create feature vectors in the learning phase, and for feature extraction in the detection phase in order to classify moments, for example, “Aha moments: that can occur for a single task in a real-time setting.

Combination of Brainwave Signals from Some Electrodes

In accordance with an exemplary embodiment, feature vectors (profile) with a combination of brainwave signals in different positions of electrodes can be better than feature vectors from a single electrode.

$$w = {\sum }_{m} {\sum }_{n} {u}_{m} * {x}_{mn}$$

where, xₘₙ is a feature vector, uₘ is a weight function depending on positions of electrodes, n is the number of trials, and m is the number of electrodes. uₘ could be learned or chosen depending on how close to a position(s) where “Aha” occurs (“Aha” could be occurs in several position of the brain). For example, the first electrode position might be on anterior cingulate cortex (ACC), and the second might be on posterior cingulate cortex (PCC). In accordance with an exemplary embodiment, some electrodes can be on anterior cingulate cortex (ACC).

Combination of Brainwave Signals in Different Frequencies

In accordance with an exemplary embodiment, feature extraction of a brainwave signal from a single electrode can work well if extracted signal is converted to a frequency domain and a combination of some frequencies/frequency bands is used.

$$w = {\sum }_{l} {\sum }_{n} {v}_{l} * {x}_{ln}$$

where, xₗₙ is a feature vector on anterior cingulate cortex (ACC), posterior cingulate cortex (PCC), or right Superior Temporal Gyrus, vₗ is a weight function depending on frequencies, n is the number of trials, and l is the number of frequencies. For example, some frequencies in gamma band, such as 30 Hz, 40 Hz, and 50 Hz, can be combined to create feature vectors of good quality. For example, some frequencies in different bands, such as 10 Hz (alpha band), 30 Hz and 40 Hz (gamma band), can be combined as well.

Combination of Brainwave Signals from Some Electrodes in Different Frequencies

Feature vectors by a combination of two methods mentioned above can be better.

$$w = {\sum }_{m} {\sum }_{l} {\sum }_{n} {v}_{l} * {u}_{m} * {x}_{mln}$$

where, xₘᵢₙ is a feature vector, v₁ is a weight function depending on frequencies, uₘ is a weight function depending on positions of electrodes, l is the number of frequencies, m is the number of electrodes, n is the number of trials.

Wavelet Features

In accordance with an exemplary embodiment, another useful type of feature that can be used is the wavelet representation, that combines both time and frequency information. Using this approach, a set of wavelet features can be computed for period around the time point of interest and for one or more electrodes. Similar to the combination of brainwave signals in different frequencies and combination of brainwave signals from some electrodes in different frequencies, the wavelet features may, or may not, be combined, and way to provide a feature vector w to be used in the classifier:

$$w = {\sum }_{l} {\sum }_{n} {v}_{l} * {y}_{ln}$$

Here, v₁ is a weigh for electrode l, and yₗₙ represents a single wavelet coefficient indexed by n for electrode 1.

Specific EEG Channels

While all channels of the EEG recording device can in principle be used to create a good feature vector, some specific channels can be more relevant for detection of mental states and creativity events. For example, channels of interest for feature extraction can include the following:

1. Anterior cingulate cortex (ACC)

2. Posterior cingulate cortex (PCC),

3. Right Superior Temporal Gyrus

4. Additional site that may be of interest: parietal, occipital, or frontal sites

In accordance with an exemplary embodiment, a non-transitory computer readable medium containing a computer program having computer readable code embodied for detecting creativity in real-time, comprising: sensing electrical activity along a scalp of a subject during a learning phase, the learning phase including presenting to the subject one or more tasks configured to generate electrical activity corresponding to cortical events that are likely to correspond to a creative type experience and cortical events that are likely to correspond to a non-creative type experience; classifying the electrical activity obtained during the learning phase to create a brainwave profile for the subject; sensing in real-time electrical activity along the scalp of the subject during a performance of one or more real-time tasks; comparing the electrical activity of the subject to previously recorded electrical activity using the brainwave profile for the subject; and classifying the electrical activity obtained during the performance of the one or more real-time tasks into the cortical events that are likely to correspond to the creative type experience and the cortical events that are likely to correspond to the non-creative type experience.

The non-transitory computer usable medium may be a magnetic recording medium, a magneto-optic recording medium, or any other recording medium which will be developed in future, all of which can be considered applicable to the present invention in all the same way. Duplicates of such medium including primary and secondary duplicate products and others are considered equivalent to the above medium without doubt. Furthermore, even if an embodiment of the present invention is a combination of software and hardware, it does not deviate from the concept of the invention at all. The present invention may be implemented such that its software part has been written onto a recording medium in advance and will be read as required in operation.

The method and system for detecting creativity in real-time as disclosed herein may be implemented using hardware, software or a combination thereof. In addition, the method and system for detecting creativity in real-time as disclosed herein may be implemented in one or more computer systems or other processing systems, or partially performed in processing systems such as personal digit assistants (PDAs). In yet another embodiment, the invention is implemented using a combination of both hardware and software.

It will be apparent to those skilled in the art that various modifications and variation can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A method for detecting creativity in real-time, the method comprising:
sensing electrical activity along a scalp of a subject during a learning phase, the learning phase including presenting to the subject one or more tasks configured to generate electrical activity corresponding to cortical events that are likely to correspond to a creative type experience and cortical events that are likely to correspond to a non-creative type experience;
classifying the electrical activity obtained during the learning phase to create a brainwave profile for the subject;
sensing in real-time electrical activity along the scalp of the subject during a performance of one or more real-time tasks;
comparing the electrical activity of the subject to previously recorded electrical activity using the brainwave profile for the subject; and
classifying the electrical activity obtained during the performance of the one or more real-time tasks into the cortical events that are likely to correspond to the creative type experience and the cortical events that are likely to correspond to the non-creative type experience.

2. The method of claim 1, comprising:
computing a Receiver Operation Characteristic (ROC) curve based on a threshold classifier applied to at least one channel within a time frame and at least one frequency band; and
computing for each of the at least one power channels an area under the curve (AUC), which represents a performance of the threshold classifier; and
choosing a channel from the at least one channel and a frequency from the at least one channel that provides a greatest area under the curve (AUC).

3. The method of claim 2, comprising
classifying the electrical activity obtained during the learning phase based on statistical thresholds, wherein the statistical thresholds are based on a desired probability of True-Positives and False-Positives.

4. The method of claim 1, comprising
detecting electrical activity of the subject on an anterior cingulate cortex (ACC), a posterior cingulate cortex (PCC), and/or or right Superior Temporal Gyrus.

5. The method of claim 1, comprising:
generating the brainwave profile by extracting features of the electrical activity from EEG data using:
a combination of brainwave signals from one or more electrodes;
a combination of brainwave signals in different frequencies; and/or
a combination of brainwave signals from one or more electrodes in different frequencies.

6. The method of claim 1, comprising:
generating the brainwave profile by extracting features of the electrical activity from EEG data using wavelets and/or Fourier coefficients.

7. The method of claim 1, wherein the learning phase comprises:
presenting specific visual stimuli for Stimuli presentation and learning signatures of creativity and specific mental states likely to lead to creative solutions to the subject to generate the electrical activity.

8. The method of claim 1, comprising:
sensing the electrical activity of the subject using an electroencephalograph (EEG) device; and
recording the electrical activity over one or more frequency bands, wherein the one or more frequency bands include electrical signals between approximately 0.1 Hz and 100 Hz.

9. The method of claim 1, comprising:
generating the brainwave profile for subject by manually recording the subject's responses in a binary format to the one or more tasks configured to generate electrical activity corresponding to the cortical events that are likely to correspond to a creative type experience and the cortical events that are likely to correspond to a non-creative type experience.

10. A system for detecting creativity in real-time, the system comprising:
a sensing device for sensing electrical activity over one or more frequency bands along a scalp of a subject, the sensing device configured to:
sense electrical activity along a scalp of a subject during a learning phase, the learning phase including presenting to the subject one or more tasks configured to generate electrical activity corresponding to cortical events that are likely to correspond to a creative type experience and cortical events that are likely to correspond to a non-creative type experience; and
sense in real-time electrical activity along the scalp of the subject during a performance of one or more real-time tasks; and
a computer device having executable instructions for:
classifying the electrical activity obtained during the learning phase to create a brainwave profile for the subject;
comparing the electrical activity of the subject to previously recorded electrical activity using the brainwave profile for the subject; and
classifying the electrical activity obtained during the performance of the one or more real-time tasks into the cortical events that are likely to correspond to the creative type experience and the cortical events that are likely to correspond to the non-creative type experience.

11. The system of claim 10, wherein the computer device is configured to:
compute a Receiver Operation Characteristic (ROC) curve based on a threshold classifier applied to at least one channel within a time frame and at least one frequency band; and
compute for each of the at least one power channels an area under the curve (AUC), which represents a performance of the threshold classifier; and
choose a channel from the at least one channel and a frequency from the at least one channel that provides a greatest area under the curve (AUC).

12. The system of claim 11, wherein the computer device is configured to:
classify the electrical activity obtained during the learning phase based on statistical thresholds, wherein the statistical thresholds are based on a desired probability of True-Positives and False-Positives.

13. The system of claim 10, wherein the sensing device is configured to:
detect electrical activity of the subject on an anterior cingulate cortex (ACC), a posterior cingulate cortex (PCC), and/or or right Superior Temporal Gyrus.

14. The system of claim 10, wherein the learning phase comprises:
presenting specific visual stimuli for Stimuli presentation and learning signatures of creativity and specific mental states likely to lead to creative solutions to the subject to generate the electrical activity; and
generating the brainwave profile for subject by manually recording the subject's responses in a binary format to the one or more tasks configured to generate electrical activity corresponding to the cortical events that are likely to correspond to a creative type experience and the cortical events that are likely to correspond to a non-creative type experience.

15. A non-transitory computer readable medium containing a computer program having computer readable code embodied for detecting creativity in real-time, comprising:
sensing electrical activity along a scalp of a subject during a learning phase, the learning phase including presenting to the subject one or more tasks configured to generate electrical activity corresponding to cortical events that are likely to correspond to a creative type experience and cortical events that are likely to correspond to a non-creative type experience;
classifying the electrical activity obtained during the learning phase to create a brainwave profile for the subject;
sensing in real-time electrical activity along the scalp of the subject during a performance of one or more real-time tasks;
comparing the electrical activity of the subject to previously recorded electrical activity using the brainwave profile for the subject; and
classifying the electrical activity obtained during the performance of the one or more real-time tasks into the cortical events that are likely to correspond to the creative type experience and the cortical events that are likely to correspond to the non-creative type experience.

16. The computer readable medium of claim 15, comprising:
computing a Receiver Operation Characteristic (ROC) curve based on a threshold classifier applied to at least one channel within a time frame and at least one frequency band; and
computing for each of the at least one power channels an area under the curve (AUC), which represents a performance of the threshold classifier; and
choosing a channel from the at least one channel and a frequency from the at least one channel that provides a greatest area under the curve (AUC).

17. The computer readable medium of claim 16, comprising
classifying the electrical activity obtained during the learning phase based on statistical thresholds, wherein the statistical thresholds are based on a desired probability of True-Positives and False-Positives.

18. The computer readable medium of claim 15, comprising
generating the brainwave profile by extracting features of the electrical activity from EEG data using:
a combination of brainwave signals from one or more electrodes;
a combination of brainwave signals in different frequencies; and/or
a combination of brainwave signals from one or more electrodes in different frequencies.

19. The computer readable medium of claim 15, comprising:
generating the brainwave profile by extracting features of the electrical activity from EEG data using wavelets and/or Fourier coefficients.

20. The computer readable medium of claim 15, comprising:
generating the brainwave profile for subject by manually recording the subject's responses in a binary format to the one or more tasks configured to generate electrical activity corresponding to the cortical events that are likely to correspond to a creative type experience and the cortical events that are likely to correspond to a non-creative type experience.
